(19) United States
(12) Patent Application Publication
(10) Pub. No.: US 2023/0320986 A1
(43) Pub. Date: Oct. 12, 2023

(54) TARGETED ANTHRACYCLINE DELIVERY SYSTEM FOR CANCER TREATMENT

(71) Applicant: TOXINVENT OÜ, Tartu (EE)

(72) Inventors: Olga TSUBRIK, Tartu (EE); Andrus TASA, Tartumaa (EE); Ain UUSTARE, Tartu (EE); Ivan OGIBALOV, Tartumaa (EE); Tambet TEESALU, Tartu (EE); Lorena Simón GRACIA, Tartu (EE); Valeria SIDORENKO, Tartumaa (EE)

(21) Appl. No.: 18/017,920

(22) PCT Filed: Jul. 29, 2021
(86) PCT No.: PCT/EP2021/071321

§ 371 Date: Jan. 25, 2023

(30) Foreign Application Priority Data
Jul. 30, 2020 (GB) 2011870.9

(51) Int. Cl. A61P 35/00; A61K 9/127; A61K 47/69; A61K 31/704; A61K 47/62

(52) U.S. Cl. CPC A61K 9/1273; A61K 31/704; A61K 47/62; A61K 47/6915; A61P 35/00

(57) ABSTRACT

The present invention relates to a drug delivery system at least comprising a drug encapsulated in a polymeric nano vesicle (polymersome), wherein the drug component is an anthracycline derivative according to the formula I wherein R¹ is selected from the group consisting of H, F, —OMe or —OEt; R² is selected from the group consisting of H, —OMe, methyl or ethyl; R³ is selected from the group consisting of H, methyl or ethyl, and R⁴ is H or a protecting group; the polymersome is formed by polymers comprising PEG, PEA, PCL, PTMC or PTMB building blocks or combinations thereof, wherein the polymersome polymers are, at least in part, functionalized by chemically attaching via a linker group L a targeting moiety, wherein the targeting moiety is selected from the group consisting of antibodies, peptides, aptamers or mixtures thereof. In addition, the present invention relates to process for the production of an anthracycline derivative loaded, targeted polymersome drug delivery system, a pharmaceutical composition comprising said drug delivery system and the use of said pharmaceutical composition for the treatment of cancer.

### FIELD OF THE INVENTION

The present invention relates to a drug delivery system at least comprising a drug encapsulated in a polymeric nanovesicle (polymersome), wherein the drug component is an anthracycline derivative according to the formula I

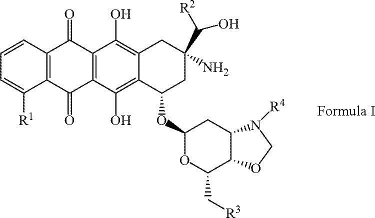

wherein R¹ is selected from the group consisting of H, F, —OMe or —OEt; R² is selected from the group consisting of H, —OMe, methyl or ethyl; R³ is selected from the group consisting of H, methyl or ethyl, and R⁴ is H or a protecting group; the polymersome is formed by polymers comprising PEG, PLA, PCL, PTMC or PTMB building blocks or combinations thereof; wherein the polymersome polymers are, at least in part, functionalized by chemically attaching via a linker group L a targeting moiety, wherein the targeting moiety is selected from the group consisting of antibodies, peptides, aptamers or mixtures thereof. In addition, the present invention relates to process for the production of an anthracycline derivative loaded, targeted polymersome drug delivery system, a pharmaceutical composition comprising said drug delivery system and the use of said pharmaceutical composition for the treatment of cancer.

### BACKGROUND

Anthracyclines, such as doxorubicin and daunorubicin, are historically among the most widely used and effective anticancer drugs in cancer treatment and have been used in cancer therapy for more than 30 years. Their mechanism of action is based on DNA intercalation causing interference in DNA synthesis and reparation and RNA production, which leads to the cell replication inhibition followed by cell death.

Structurally, anthracyclines are tetracyclic molecules with an anthraquinone backbone bound to a sugar through a glycosidic linkage. The four-ring structure intercalates between DNA and the sugar interacts with adjacent base pairs in the minor groove. The intercalation into DNA forms a stable complex between anthracycline, DNA, and topoisomerase II, thus, inhibiting the action of topoisomerase II and impeding the reparation of DNA. The mechanism of action of anthracyclines includes also the formation of toxic reactive oxygen species generated by the quinone moiety.

Although anthracyclines are effective against several cancer types, their use is limited by their cardiotoxicity. Extensive research has been done to develop synthetic anthracyclines with a high therapeutic index as well as delivery systems that decrease their side effects. PEGylated-liposomal doxorubicin (Caelyx) is used in the treatment of breast and ovarian cancer, multiple myeloma, and Kaposi’s sarcoma, and has shown improved cardiac safety without compromising the doxorubicin’s anticancer efficacy. Despite the reduced cardiotoxicity of liposomal anthracyclines compared with the free drug, in some cancer types such as metastatic breast cancer, the anticancer efficacy is not significantly higher. Moreover, skin toxicity and mucositis, among others, are common side effects of liposomal encapsulated doxorubicin.

Therefore, there is an urgent need for developing more potent chemotherapeutics as well as tumor specific delivery systems, being able to decrease the side effects of drug administration. One approach includes the use of drug nanocarriers, such as liposomes, wherein the increased accumulation in the tumor is mainly based on the enhanced permeability and retention (EPR) effect. Unfortunately, the EPR effect is highly heterogeneous and depends on the tumor type and stage.

The topic of drug delivery is also mentioned in the patent literature.

US 2008 181 939 A1 for instance discloses a hydrolysis-triggered, controlled release polymersome nano-delivery system for delivering an cytotoxic, anticancer therapeutic active agent to a cell, the system comprising: at least one hydrolytically degradable, hydrophobic block copolymer to effect controlled polyester chain hydrolysis in the membrane, such that when combined with hydrophilic PEO, the PEO volume fraction (fEO) and chain chemistry control encapsulant release kinetics from the copolymer vesicles and polymersome carrier membrane destabilization; a stable, purely synthetic, self-assembling, controlled release, polyethylene oxide (PEO)-based polymersome vesicles having a semi-permeable, thin-walled, amphiphilic, high molecular weight PEO-based block copolymer encapsulating membrane, having a desired controlled release rate for releasing the anticancer therapeutic encapsulant; which when blended in aqueous solution the at least one hydrophilic PEO-block copolymer together with the at least one inert, hydrophobic PEG-block copolymer form amphiphilic high molecular weight PEO-based polymersomes having the desired controlled release rate of the at least one anticancer active agent encapsulant contained therein, and encapsulated therein a cytotoxic anticancer therapeutic active agent.

Furthermore, US 2011 027 347 A1 describes a polymersome comprising one or more bioactive agents, wherein the polymersome is derived from a specific polymer comprising the formula XY₂, wherein X comprises a hydrophilic group and Y comprises a hydrophobic group.

In addition, US 2017 002 7868 A1 discloses a liposomal composition for treatment of cancer, comprising: a targeted PEGylated liposome, wherein the targeted PEGylated liposome is targeted by P15 molecules ranging from 25 to 100, wherein the P15 molecules are P15 peptide with a sequence of H-Cys-Gly-Gly-Gly-Pro-Pro-Leu-Ser-Gln-Glu-Thr-Phe-Ser-Asp-Leu-Trp-Lys-Leu-Leu-OH, and wherein the targeted PEGylated liposome is loaded with doxorubicin.

Nevertheless, besides the already existing solutions in the field of targeted drug delivery systems there still exists the need for further solutions, being able to provide an efficient targeted delivery of potent drugs to the tissue of interest.

Therefore, it is the task of the invention at hand to overcome, at least in part, the drawbacks of the state of the art. Especially, it is a task of the invention to provide a potent drug delivery system, wherein the cytotoxicity of the drug is improved, unwanted systemic side effects are minimized and high drug levels in cancer cells are achieved.

The above-mentioned task is solved by a drug delivery system comprising the features according to the independent claim 1. In addition, the task is further solved by a process for the production of an anthracycline derivative loaded, targeted drug delivery system, a pharmaceutical composition comprising said delivery system and the inventive use of the delivery system in the treatment of cancer according to the features of the independent claims, respectively.

Preferred embodiments of the invention are also defined by the features of the dependent claims, by features disclosed in the description and in the figures, wherein a feature aggregation of separated parts is within the scope of the invention, unless explicitly excluded.

### BRIEF DESCRIPTION OF THE INVENTION

Above mentioned problem is solved by a drug delivery system at least comprising a drug encapsulated in a polymeric nanovesicle (polymersome, PS), wherein the drug component is an anthracycline derivative according to the formula I

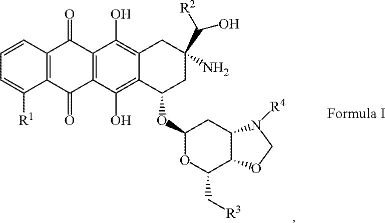

wherein R¹ is selected from the group consisting of H, F, —OMe or —OEt; R² is selected from the group consisting of H, —OMe, methyl or ethyl; R³ is selected from the group consisting of H, methyl or ethyl, and R⁴ is H or a protecting group; and wherein the polymersome is formed by polymers comprising PEG, PLA, PCL, PTMC or PTMB building blocks or combinations thereof, wherein the polymersome polymers are, at least in part, functionalized by chemically attaching via a linker group L a targeting moiety, wherein the targeting moiety is selected from the group consisting of antibodies, peptides, aptamers or mixtures thereof.

Surprisingly, it has been found that the anthracycline according to formula I comprises several synergistic advantages in combination with the above described targeted polymersome (PS) delivery system. The specific anthracyclines exhibit, compared to doxorubicin, higher anticancer effectivities against prostate carcinoma and melanoma cells. Furthermore, the encapsulation results in a biocompatible and biodegradable polymersomes, wherein the encapsulation is achieved with high encapsulation rates. The delivery system can be tailored with respect to the target cells by the choice of the targeting moiety, e.g. tumor penetrating peptides (TPPs), or other classes of affinity targeting ligands and the system efficiency can be adapted flexibly, for instance, by alteration of the tumor penetrating peptide density on the PS. The homing system is generally able to increase the drug toxicity in cells expressing the “right” receptor and, therefore, the anticancer efficacy is increased and the systemic drug side effects are reduced. Especially, the targeted delivery is able to prevent unwanted drug accumulation in the heart in vivo, avoiding undesired side effects of anthracyclines such as cardiotoxicity. Furthermore, the homing-functionalized delivery system showed very fast tumor penetration, suggesting applications in tumor detection and imaging. Such synergistic advantages are surprising, because the in vivo, in vitro effectivity depends on a complex interplay between the drug chemistry and the delivery-system. Encapsulation at high drug loadings and the resulting stability of the loaded delivery system during storage and in in vivo environments are difficult to predict. Furthermore, the stability and targetability of the delivery system has to be tailored, in order to assure only minor drug release during delivery, fast and specific targeting, complete internalization in the target cell and fast decomposition in the target. Therefore, at several stages during delivery small but significant changes in the chemical surrounding have to be able to disturb the stability equilibrium between drug and PS, resulting in the desired action of the drug.

The drug delivery system at least comprises a drug encapsulated in a polymeric nanovesicle. The disclosed system comprises at least two different kinds of molecules. On the one hand a drug, i.e. a substance that causes a change in a human or animal organism’s physiology or psychology when consumed and a vesicle surrounding or encapsulating the drug. This means, that the drug is either buried in the inside of a vesicle or forms part of the vesicle bilayer, wherein the bilayer wall is formed by polymers. The vesicle dimensions are in the sub-micron range, e.g. the vesicles can be sphere-like shaped and may comprise a diameter in the range of larger or equal 50 nm and smaller or equal 500 nm.

The drug component of the delivery system is an anthracycline derivative according to the formula I

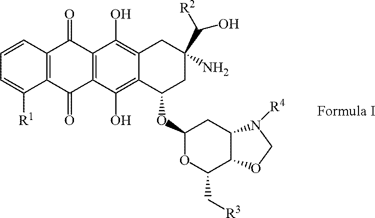

wherein R¹ is selected from the group consisting of H, F, —OMe or —OEt; R² is selected from the group consisting of H, —OMe, methyl or ethyl; R³ is selected from the group consisting of H, methyl or ethyl, and R⁴ is H or a protecting group. This new 9-aminoanthracycline derivative comprises a hydroxyl group at the C13 position and an oxazolidine cycle in the C-3′, C4′ of the daunosamine moiety. These 9-aminoanthracyclines are less cardiotoxic compared to other anthracyclines, and the cytotoxic effect of anthracyclines is further increased by attaching a methylene or an ether group to the amino and hydroxyl groups (between the respective nitrogen and oxygen atoms) of the 1,2-aminoalcohol moiety of the daunosamine. The five-membered cycle is covalently binding to DNA in the target cell, forming a drug-DNA adduct. The stability of the oxazolidine cycle in aqueous medium can be tailored by attaching a protection group at the nitrogen atom in the 5-membered cycle. A possible protection group may be linked by a carbamate bond. A suitable protection group can be acetyloxyalkyl carbamate or similar protection groups. Under certain physiological conditions, for instance by shifting the pH or esterase induced, the protecting group is hydrolyzed leading to an exposure of the reactive oxazolidine cycle. The oxazolidine cycle can react with DNA forming an anthracycline-DNA adduct.

Anthracyclines are among the most effective anticancer therapeutics and are effective against a wide range of cancer types; however, cardiotoxicity limits their dosing and exposes the patient to cardiovascular morbidity and mortality. Development of drugs and delivery systems with higher toxicity against tumor cells and less side effects are necessary to increase the therapeutic index of current cancer treatments. 9-aminoanthracyclines, such as amrubicin, are less cardiotoxic than other anthracyclines. In the metabolism of most anthracyclines the enzymatically reduction of the C-13 carbonyl group to a hydroxyl group takes place. In the case of amrubicin, the corresponding metabolite, Amrubicinol, is 5-50 times more potent than the parent drug. Intracellular reduction of anthracyclines also forms free radicals that can oxidize other molecules in the cell producing formaldehyde, which in turn reacts with amino group(s) present in the anthracycline, forming a drug-DNA adduct. For adduct formation, formaldehyde first reacts with the 3′-amino of daunosamine in the anthracycline forming an activated Schiff base which is then able to form an aminal (N-C-N) linkage to the exocyclic amino group of guanine residues. This mechanism of cytotoxicity can be facilitated by the formation of the oxazolidine cycle. The design of the proposed anthracycline derivative was based on:
1) the reduced cardiotoxicity of 9-aminoanthracyclines;
2) the increase of the potency by reducing the C-13 carbonyl group to hydroxyl group; and
3) the increase of the cytotoxicity by forming an oxazolidine cycle.

When the anthracycline derivative penetrates the cell, the protecting groups are hydrolyzed by esterases in the cytosol, exposing the reactive oxazolidine cycle. The four-ring structure of the anthracycline derivative can intercalate into the DNA and the oxazolidine cycle covalently binds to guanine via the methylene carbon thus blocking molecular processes of DNA. Esterases like carboxylesterases are overexpressed in some tumor types, making the selected anthracycline derivative a better tumor selective drug compared to other anthracyclines, such as DOX.

The PS is formed by polymers comprising PEG, PLA, PCL, PTMC or PTMB building blocks or combinations thereof. It has been found that PS formed from or comprising the above mentioned polymeric blocks do show favorable characteristics with respect to drug loading capacity, stability and kinetics of drug release. It is assumed that the hydrophilic/hydrophobic contributions of the blocks are in the right range in order to interact with the functional groups or ring structures of the inventively used anthracycline derivative. The blocks can be blocks comprising two or more of the mentioned monomers. The meaning of the abbreviations is known to the skilled artisan, for instance PEG means polyethylenglycole, PLA (polylactic acid or polylactide), PCL (polycaprolactone); PTMC poly(trimethylene carbonate). The PEG-Block may for instance comprise between 2000 and 10000 repetition units and the other blocks may, for instance, comprise between 5000 and 40000 repetition units, preferably the PEG-Block may comprise between 4000 and 7000 repetition units and the other blocks may preferably comprise between 8000 and 20000 repetition units.

The polymersome polymers are, at least in part, functionalized by chemically attaching via a linker group L a targeting moiety, wherein the targeting moiety is selected from the group consisting of antibodies, peptides, aptamers or mixtures thereof. In order to generate a homing or targeted PS, all or a fraction of the polymers comprise a chemical moiety, wherein the chemical moiety is covalently linked by the linker group L to the polymers building the PS. The moiety in general is biological active in a sense, that the moiety interacts biologically with the target cell, e.g. by attaching to the target cell or by triggering internalization into the target cell. Therefore, the chemical moiety induces a preferred interaction between the drug delivery system and the target cell type. Possible moieties can be selected from above given list, wherein the right moiety type can be selected as a function of the target. A mixture may comprise two or more different targeting moieties on the same polymersome. Aptamers for instance are oligonucleotide or peptide molecules that bind to a specific target molecules or cells. A target molecule can for instance be a cell surface receptor. An antibody is a protein, wherein the protein structure also enables a specific binding of the protein to target structures. Suitable peptides can for instance be TPPs. The targeting moieties can be attached to the polymers by suitable functional groups of the targeting moiety. The specific functional groups are known to the skilled artisan. A suitable degree of polymer functionalization is a function of the targeting moiety size and the overall PS stability. Possible ratios of functionalized to not functionalized polymers can be in larger or equal to 1% and smaller or equal to 100%.

Within a preferred aspect of the drug delivery system the polymersome polymers are, at least in part, functionalized by chemically attaching via a linker group L tumor penetrating peptides selected from the group consisting of CendR peptides, iRGD (CRGDKGPDC), LyP-1 (CGNKRTRGC), RPAR (RPARPAR), TT1 (CKRGARSTC), LinTT1 (AKRGARSTA), iNGR (CRNGRGPDC), tLyp-1 (CGNKRTR) or precursors thereof. In order to generate a homing or targeted PS, all or a fraction of the polymers comprise a peptide group, wherein the peptide group is covalently linked by the linker group L to the polymers building the PS. CendR peptides, for instance, enhance the permeability of tumor blood vessels and tumor tissues via binding to neuropilin-1 (NRP-1) a transmembrane glycoprotein. The CendR peptides comprise the sequence (R/KXXR/K). iRGD peptides target tumor fibroblasts or tumor cells and comprises a CRGDKGPDC sequence. LyP-1 peptides target tumor endothelial cells, macrophages, tumor lymphatics, tumor cells and comprise a sequence of CGNKRTRGC. RPAR peptides target NRP-1 expressing cells (tumor endothelial cells, macrophages, tumor lymphatics, tumor cells) and comprise a sequence of RPARPAR. TT1 peptides target tumor endothelial cells, macrophages, tumor lymphatics, tumor cells and comprise a sequence of CKRGARSTC. LinTT1 peptides target tumor endothelial cells, macrophages, tumor lymphatics, tumor cells and comprise a AKRGARSTA sequence. iNGR peptides target tumor endothelial cells or other cells in in tumors and comprise a CRNGRGPDC sequence. tLyp-1 peptides target tumor endothelial cells or other NRP-positive cells in tumors and comprise a CGNKRTR sequence. Besides the given sequences it is possible that the peptides are attached in the form of peptide precursors to the PS, wherein the precursor may comprise further functional or non-functional groups, wherein the groups may be removed in vivo from the peptide fragment prior to attachment to the target cell.

Within a preferred embodiment of the drug delivery system the polymersome may comprise di-block PEG-copolymers, wherein the second block is selected from the group consisting of PLA, PCL, PTMC, PTMBP. For PS stability, drug loading capacity and enhanced in vivo half-life it has been found useful to build the PS from di-block copolymers at least comprising a PEG-block. The other block can be selected from the above given group and the di-block copolymer can be a biodegradable polymer, comprising an increased stability in combination with the inventive anthracycline under in-vivo conditions and an accelerated breakdown in the tumor cell.

In a preferred aspect of the drug delivery system the polymersome may consist of PEG-PCL diblock-copolymers, wherein the weight ratio of the different polymer blocks, calculated as PEG-segment weight divided by PCL-segment weight, is larger or equal 0.1 and smaller or equal 5. The encapsulation of the anthracycline derivative in above described PS has been proven highly effective in in vivo and in vitro tests against tumor cells. Without being bound to the theory it is believed, that the interaction of the drug with the di-block chains results in an improved stability, reducing the risks of drug release prior to entering the target cell. Furthermore, besides the improved stability also high drug loads can be incorporated in the vesicles based on the favorable drug-polymer interaction. Lower ratios can be disadvantageous because the PS stability can be decreased, higher ratios may be disadvantageous because the PS may get to hydrophobic, thus reducing PS flexibility and solubility/stability in in vivo media.

In a preferred aspect of the drug delivery system in formula I R¹ = H, R², R³ = H, and R⁴ can be a protecting group. This substitution pattern of the anthracycline has been found to show an improved cytotoxicity and an improved PS encapsulation stability. The latter may be based on the favorable interaction of the methyl groups to the polymeric PS blocks, while the physiologic effect can be attributed to the substitution and the overall ring structure, comprising an oxazolidine cycle, wherein the latter exhibits better interaction with the DNA-structures of the target cells.

Within a further preferred characteristic of the drug delivery system R⁴ can be acetyloxymethyl carbamate. It has been found useful to incorporate into the PS the anthracycline comprising a carbamate protected oxazolidine cycle. It appears that protection is not only changing the stability of the oxazolidine cycle itself, but also comprises favorable interactions with the delivery system by forming increased interactions with the polymeric blocks. The R⁴ may comprises the general formula RR′N—CO—O—CHR″—O—CO—R′”’, wherein R” may be H or Me and COR”’may be acyl. Based on the substitution pattern of the protection groups also the hydrolysis rate can be tailored in the tumor cell surrounding.

In another, preferred embodiment of the drug delivery system the group L can be maleimide. In order to achieve a functional attachment of the peptides or peptide precursors to the PS maleimide has been found beneficial. The attachment can be performed very selectively and the overall PS structure, drug loading capacity and integrity is not disturbed. The attachment of the peptide to the maleimide is favorably achieved via a thioether bond between the maleimide and cysteine amino-acids of the peptides.

In a preferred embodiment of the drug delivery system the molar ratio of peptide modified polymer chains to the total number of polymers chains in the polymersome, calculated as number of peptide-modified polymer chains divided by total number of polymer chains, can be larger or equal to 0.01 and smaller or equal to 0.4. In order to enhance PS stability and the efficiency of internalization of the PS into the tumor target cell, above mentioned molar ratios have been found favorable. The overall PS solubility and stability, as given by the structure of the block-polymers, is not significantly altered in that range. Furthermore, lower ratios can be disadvantageous because the targeted interaction with the surface receptors of the tumor cells is insufficient. The ratio can be assessed by fluorescence measurements of fluorescence dye labeled peptides.

In a preferred aspect of the drug delivery system the molar ratio of peptide modified polymer chains to the total number of polymers chains in the polymersome, calculated as number of peptide-modified polymer chains divided by total number of polymer chains, is larger or equal to 0.05 and smaller or equal to 0.1. In order to preserve the fundamental stability, density and size characteristics of the anthracycline loaded PS, also keeping the desired dissolution and drug release behavior of the targeted PS, above captioned ratio has been found useful. The PS show an improved cellular binding, internalization efficiency and size range compared to PS comprising out of range ratios.

Within a further preferred embodiment of the drug delivery system according the concentration of the drug in the polymeric nanovesicle can be larger or equal to 20 µM and smaller or equal to 500 µM. The combination of the anthracycline and the PS enables the encapsulation of higher drug amounts compared to other encapsulation systems. The stability of PS especially comprising the above mentioned block-copolymers enables the encapsulation of such high amounts, without or with only a small release prior to internalization of the PS in the target cells. Such stability is unusual, because the drug load should also influence the polymer packing and the polymer interaction of the PS wall.

Within another preferred characteristic of the drug delivery system the average hydrodynamic diameter of the drug loaded polymeric nanovesicle can be larger or equal to 80 nm and smaller or equal to 125 nm. In order to find the most efficient balance between PS volume/drug load, stability under in vivo conditions and internalization efficiency above mentioned PS size range has been found beneficial. Larger sizes may reduce the internalization efficiency and overall stability of the drug in the PS and smaller PS-sizes may negatively affect the possible drug amount in the PS. The hydrodynamic diameter can be obtained by dynamic light scattering as described in the experimental part.

In a further preferred embodiment of the drug delivery system the polydispersity index of the drug loaded polymeric nanovesicle can be larger or equal to 0.01 and smaller or equal to 0.25. It has been found beneficial for the internalization efficiency and stability of the drug loaded PS to use PS with above captioned polydispersity. Based on the homogeneous and narrow PS size distribution the systemic drug release can be reduced.

In addition, it is within the scope of the invention at hand to disclose a process for the production of an anthracycline derivative loaded, targeted polymersome drug delivery system according to any one of the preceding claims, characterized in that the drug according to formula I is encapsulated in the polymeric nanovesicle by a thin-film hydration step. Especially thin-film hydration has been found useful for building the inventive system including the specific anthracycline drug and the specific polymer selection. It is possible to achieve polymer size distribution in the “right” polymer size range, comprising a low level of polydispersity, only. In addition, it seems that especially the anthracycline derivatives are stably incorporated into the PS without significant amounts of only “loosely” incorporated or attached drug molecules.

Within a preferred aspect of the process the drug loaded polymeric nanovesicle can be subjected in a further step to a size-exclusion chromatographic step. In order to achieve a very narrow size distribution and in order to assure very low levels of not properly incorporated drug, a size exclusion step has been found very useful. A very stable and narrow size distribution is achieved, wherein the drug comprises a very homogeneous elution profile upon change of the chemical surrounding.

It is further within the scope of the invention to disclose a pharmaceutical composition comprising the inventive drug delivery system in a pharmaceutically acceptable solvent. The inventive drug delivery system can easily be dissolved in a number of pharmaceutically acceptable solvents, thus forming a stable suspension. For the advantages of the pharmaceutical composition it is explicitly referred to the advantages of the inventive drug delivery system. Suitable acceptable solvents may for instance be selected from the group consisting of PBS, saline or mixtures thereof.

Furthermore, it is within the scope of the invention to disclose the use of the inventive pharmaceutical composition for the treatment of cancer. The inventive pharmaceutical composition including the inventively drug delivery system comprising the inventive anthracycline drug comprises superior anti-tumor efficacy in in vitro and in vivo experiments. The better effects are, at least in parts, based on the higher toxicity of the drug compared to state of the art anthracyclines. Based on the synergistic combination of drug and delivery system it is possible to provide a better biocompatibility and less side effects compared to the state of the art delivery systems. Therefore, a safe and very efficient targeted anti-tumor vehicle is provided by the invention.

### EXPERIMENTAL EXAMPLES

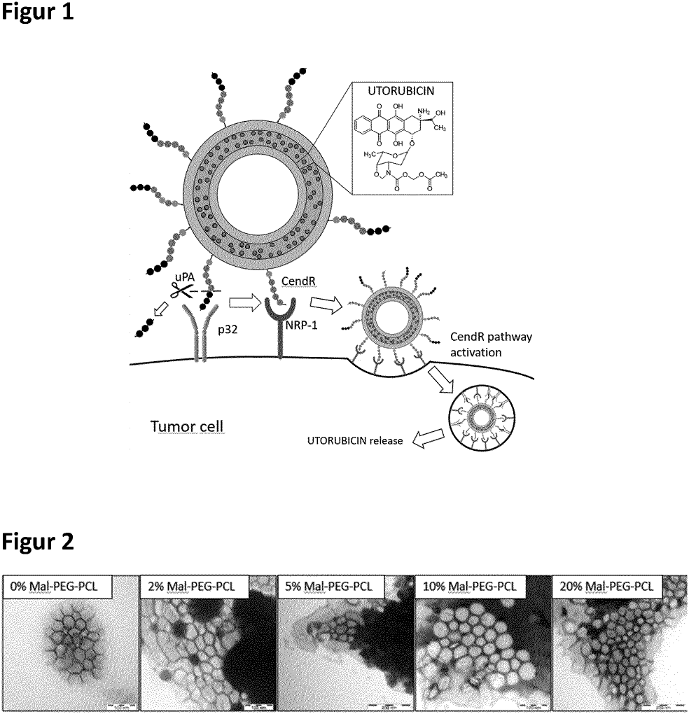

A possible mode of action of the inventive drug delivery system is schematically displayed in FIG. 1.

For all experiments the following anthracycline derivative (UTO) was used:

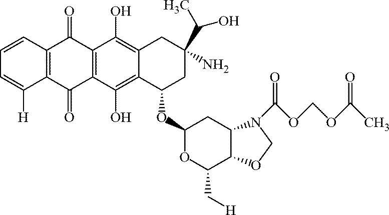 [SMILES: CC(=O)OCOC(=O)N1CO[C@@H]2[C@H](C)O[C@@H](O[C@H]3C[C@](N)(C(C)O)Cc4c(O)c5c(c(O)c43)C(=O)c3ccccc3C5=O)C[C@@H]21]

The anthracycline derivative comprises with respect to formula I the following substitution pattern: R¹ = H; R² = CH₃; R³ = H and R⁴ = protecting group (i.e. acetyloxymethyl carbamate).

### 1. Drug Synthesis

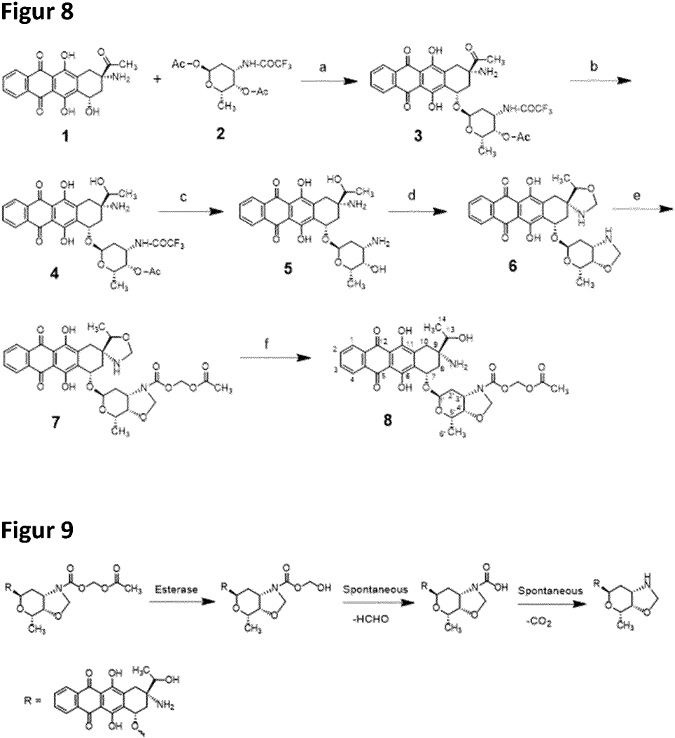

The UTO synthesis is performed according to the scheme as depicted in FIG. 8. A possible mechanism of in vivo de-protection of the protected drug is shown in FIG. 9.

Amrubicinone (1) was glycosylated with 1,4-di-O-acetyl-N-trifluoroacetyl-β-L-daunosamine (2) in presence of trimethylsilyl trifluoromethanesulfonate. After quenching of the reaction, the product was purified by column chromatography on silica gel (eluent diethyl ether/ethyl acetate). The carbonyl group of compound 3 was reduced using 2.1 equivalents of sodium triacetoxyborohydride in ethanol, the crude product was extracted in diethyl ether and purified by column chromatography on silica gel (eluent dichloromethane/methanol). Compound 4 was deprotected - N-trifluoroacetyl and O-acetyl groups from the L-daunosamine part were cleaved using lithium hydroxide (10 equivalents) in tetrahydrofuran/methanol/water mixture. The reaction mixture was neutralized to pH 8.2 and the crude product was separated by extraction. The crude product was further purified by column chromatography using lower phase of chloroform/methanol/aqueous ammonia mixture and chloroform as eluents. The purified compound 5 was reacted with 1.9 equivalents of paraformaldehyde in dry chloroform for 3 days. Unreacted compound 5 was separated by filtration through 0.45 µm pore filter, the obtained solution was concentrated and triturated with diethyl ether to obtain compound 6. The product was characterized by Nuclear Magnetic Resonance (NMR).

Synthesis of compound 8: 100 mg of compound 6 (0.19 mmol) was dissolved in 6 mL of dry dimethyl formamide and 49 mg (1 equivalent, 0.19 mmol) of 4-nitrophenyl-(acetyloxy)-methylcarbonate was added. The mixture was stirred for 26 h at room temperature under argon atmosphere. After this period, reaction mixture was partially concentrated (to ~1 mL) at room temperature under reduced pressure, mixed with 6 mL of solution of acetic acid (1%) in acetonitrile:water (1:1) and stirred for 2 h to hydrolyze the unreacted oxazolidine cycle. The obtained mixture was purified by preparative HPLC (Column Luna C18(2) Axia 27.2×250 mm, eluent system water/acetonitrile). A total of 22 mg of conjugate 8 was separated. The structure of the product was confirmed by NMR and High Resolution Mass Spectrometry (HRMS, calculated MW 627.2185, found MW 627.2182).

### 2. Synthesis of Drug Delivery Systems

Polyethylene glycol-polycaprolactone (PEG₅₀₀₀-PCL₁₀₀₀₀; M_{w} 5,000 and 10,000 respectively) (PEG-PCL), Fluorescein-PEG-PCL (FAM-PEG-PCL), and maleimide-PEG₅₀₀₀-PCL₁₀₀₀₀ (mal-PEG-PCL) were mixed and dissolved in 0.5 mL acetone. The total amount of polymer was 5 mg. Different percentages of mal-PEG-PCL were used (0; 2; 5; 10; and 20%), and all the polymersomes (PS) samples contained 5% of FAM-PEG-PCL polymer. The acetone was evaporated with nitrogen flow forming a thin polymeric film on the wall of the glass vial (Sigma-Aldrich, Germany). Next, the film was hydrated with 0.4 mL of PBS pH 7.4 previously purged with nitrogen flow, heated for 30 seconds in 65° C. water bath and sonicated for 30 seconds. The heating and sonication steps were repeated until the PS were formed and polymer aggregates were not observed in the suspension. After that, 4 equivalents of Cys-RPAR peptide with respect the mal-PEG-PCL were dissolved in 0.1 mL of PBS and added to the PS suspension. The sample was sonicated for additional 10 minutes, mixed in the shaker for 3 h at RT and kept overnight at 4° C. The final volume of PS samples was 0.5 mL and total polymer concentration 10 mg/mL.

For the encapsulation of the drug inside PS, 50 nmols of drug were dissolved in 100 µL of acetone and added to the polymers dissolved in acetone (total amount of polymer was 5 mg). The acetone was evaporated to form the polymer/drug film and the PS were formed as described above.

PS were purified by size exclusion chromatography. Agarose beads with a diameter of 45-165 µm (Sephadex 4B gel) were used as stationary phase. The height of the Sephadex gel in a column was 8 cm (volume 25.13 mL). The PS sample was eluted with PBS pH 7.4.

The average hydrodynamic diameter of PS was measured with dynamic light scattering (DLS) by using a Zetasizer Nano ZSP (Malvern, USA). PS samples were diluted with PBS pH 7.4 to 1 mg/mL. Samples were scanned for 10 seconds at 173°. The results represent and average over 10 runs. Measurements were repeated 3 times and averaged. Zeta potential was measured using Zetasizer Nano ZSP (Malvern, USA) at 0.2 mg of polymer/mL in NaCl 10 mM, performing 50 runs per sample. For transmission electron microscopy (TEM), PS samples were diluted in mQ water (0.5 mg/mL) and transferred onto copper grids for 1 min, stained with 0.75% phosphotungstic acid (pH 7) for 20 sec, air-dried, and visualized using Tecnai 10 TEM (Philips, Netherlands).

The amount of encapsulated drug was quantified using a Nanodrop 2000c UV-VIS spectrophotometer (Thermo Scientific, USA). For drug quantification, serial dilutions of drug in MeOH:water 1:1 were prepared and the absorbance at 490 nm was measured. Using gathered data, the linear trend line was constructed in MS Excel program and the formula was further used to evaluate the concentration of drug inside PS.

The percentage of FAM-PEG-PCL in the PS samples was quantified by fluorimetry. First, a calibration curve of FAM-Cys was prepared in MeOH:PBS 1:1 and the fluorescence at 480 nm/535 nm was measured using Victor X5 Multilabel Microplate Reader (Perkin Elmer, USA). PS samples (25 µL) were mixed with 25 µL of MeOH and the fluorescence was measured to calculate the percentage of FAM-PEG-PCL in the PS composition. The FAM-PEG-PCL percentage in the PS composition was 4.9 ± 0.3.

To estimate the peptide amount on the PS with optimum peptide density, PS were formed using 20% of Mal-PEG-PCL and 80% of PEG-PCL and FAM-Cys-RPAR peptide was conjugated to the PS as described above. The standard curve of FAM-Cys-RPAR was prepared in PBS and the fluorescence was measured by fluorimetry at 480 nm/535 nm. PS functionalized with FAM-Cys-RPAR peptide (25 µL) were mixed with 25 µL of MeOH and the fluorescence was measured to calculate the percentage of FAM-RPAR-PEG-PCL in the PS composition. The FAM-peptide-PEG-PCL percentage with respect the total polymer amount was 6%.

### 3. Characterization of the Drug Delivery System

### 3.1 Peptide Density

For testing the effect of the overall peptide density, polymersomes comprising different RPAR densities on their surface were prepared. The density was varied by using different proportions of maleimide-PEG-PCL (0; 2; 5; 10 and 20%) relative to whole amount of co-polymer used for synthesis. The maleimide group amount determines the maximum achievable peptide density, as peptide conjugation occurs through formation of thioether bond between the cysteine thiol group of the peptide and the maleimide group of the copolymer. All PS were prepared by the method described above. PS were functionalized with Cys-RPAR peptide and contained 5% of FAM-PEG-PCL as a fluorescent-label.

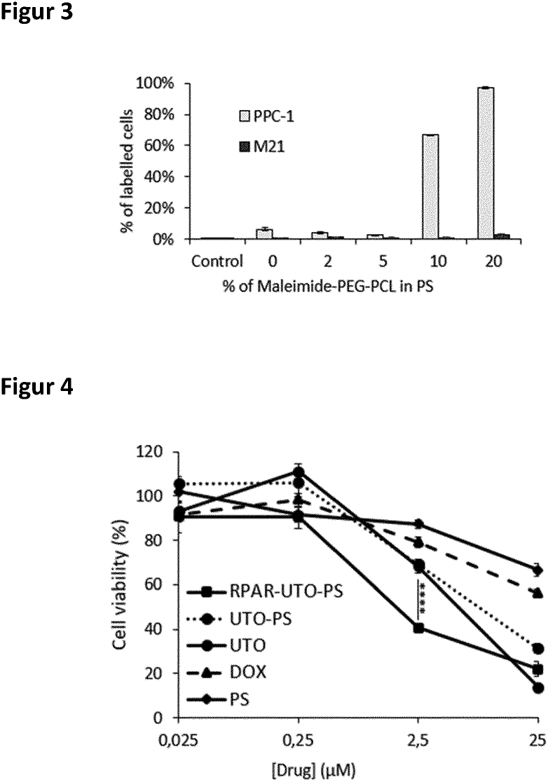

The hydrodynamic diameter of the different PS samples was measured by dynamic light scattering. The average PS diameter was 105 ± 12 nm and the polydispersity index (PDI) was 0.19 ± 0.02. Transmission electron microscopy showed that all the FAM-labeled RPAR-PS (RPAR-FAM-PS) samples were homogeneous comprising spherical vesicles (FIG. 2).

In addition, the RPAR density effect on the PS surface was evaluated with respect to the best tumor cell targeting using PPC-1 and M21 tumor cells. PPC-1 cells, derived from human primary prostate cancer cells, comprise, in comparison to healthy cells, elevated expression of NRP-1 receptors. On the contrary, M21 cells, derived from human melanoma cells, are lacking NRP-1. These cell lines provide a useful tool for studying the specific binding and internalization of RPAR-targeted PS to NRP-1 expressing cells.

Both cell lines were incubated with the different RPAR-FAM-PS samples for 1 h and the cellular binding and internalization were measured using flow cytometry. There was specific binding of RPAR-FAM-PS to PPC-1 cells and the peptide increase on PS surface resulted in higher internalization in PPC-1 cells (FIG. 3). PS formed with 20% maleimide-PEG-PCL showed the highest uptake by PPC-1 cells, approximately 100% of the labeled cells. In contrast, the binding of RPAR-FAM-PS to M21 cells was very low and independent from peptide density, confirming the dependency of cell binding and internalization on the peptide-receptor interaction.

In addition, the RPAR-FAM-PS uptake by PPC-1 and M21 cells was tested with fluorescence confocal microscopy. After 1 h incubation, the signal representing RPAR-FAM-PS was detected only in PPC-1 cells whereas M21 cells did not show PS uptake. Confirming the results from flow cytometry, significantly higher RPAR-FAM-PS signal was detected in PPC-1 cells incubated with PS formed using 20% maleimide-PEG-PCL. Notably, when PPC-1 cells were incubated with PS having 20% of maleimide-PEG-PCL, the corresponding fluorescent signal was seen inside the cells, indicating the successful cell penetration of RPAR-FAM-PS. Based on these findings, further PS were synthesized using 20% of maleimide-PEG-PCL.

### 3.2. Encapsulation Efficiency

For assessment of the UTO encapsulation efficiency the above depicted anthracycline derivative was encapsulated in a PS using a thin-film hydration method. After encapsulation the sample was purified by size exclusion chromatography to remove non-encapsulated drug. The morphology and hydrodynamic diameter of loaded RPAR-functionalized PS (RPAR-UTO-PS), UTO-loaded non-targeted PS (UTO-PS), and “empty” PS (PS) were similar for all samples and in the range of 100 +- 12 nm and a PDI of 0.15 +- 0.06 indicating a very homogeneous PS diameter. The UTO concentration in UTO-PS samples was approx. 50 µM with an encapsulation efficiency (EE) of 80%. The retention of UTO in the PS membrane, probably based on its matching hydrophobic character, resulted in a higher EE compared to encapsulation efficiency of Doxorubicin·HCl (1% EE).

### 3.3. Cytotoxicity

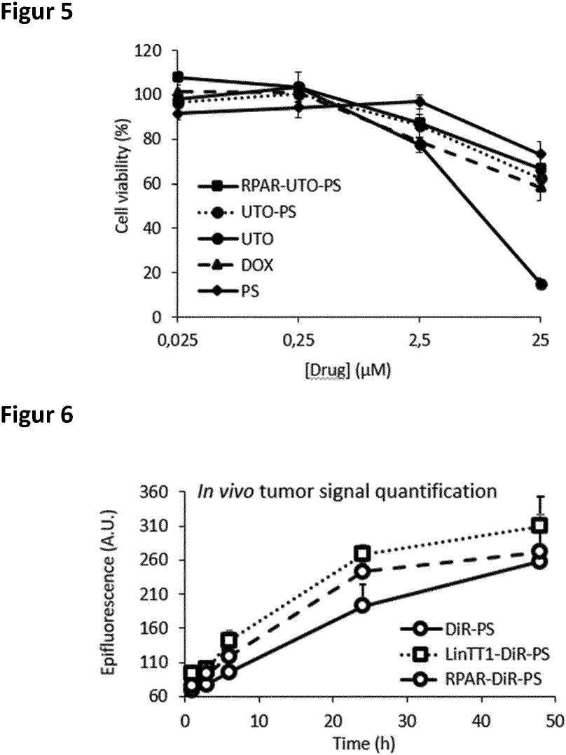

For assessment of the UTO cytotoxicity the cytotoxicity of RPAR-UTO-PS, UTO-PS, PS, free UTO, and free DOX in cultured PPC-1 (FIG. 4) and M21 (FIG. 5) cells after 30 min treatment over a range of drug concentrations was tested. Free UTO was significantly more toxic compared to free DOX in PPC-1 cells at concentrations of 2.5 µM (cell viability 68% vs 87% respectively) and 25 µM (14% vs 56% respectively). In M21 cells, free UTO also showed a higher toxicity compared to DOX at concentration of 25 µM (15% vs 58%). In NRP-1 positive PPC-1 cells RPAR-UTO-PS were significantly more toxic than UTO-PS at a concentration of 2.5 µM of UTO (41% of cell viability versus 70%). NRP-1 negative M21 cells showed significantly less viability when treated with free UTO at a concentration of 25 µM, confirming that TPP-PS penetration and thus toxicity in tumor cells depends on the peptide binding to NRP-1. Moreover, RPAR-UTO-PS showed higher toxicity compared to free UTO in PPC-1 cells at 2.5 µM drug concentration, demonstrating that the internalization triggered by the CendR peptide is more efficient compared to cell internalization of the free drug at that concentration.

### 3.4. In Vivo Testing - Homing Capabilities

To evaluate the ability of PS targeted with TPPs for specific drug delivery to tumors in vivo, the tumor accumulation of PS labeled with the dye DiR in an orthotopic TNBC model was used. DiR is a hydrophobic molecule with near-infrared (NIR) absorption and emission spectrum, providing a useful tool for whole-body imaging. NIR-light is able to penetrate into tissues whilst having minimal background interference in that region.

LinTT1-, RPAR-targeted, and nontargeted PS encapsulating DiR (LinTT1-DiR-PS, RPAR-DiR-PS, and DiR-PS) were prepared. The PS were spherical with an average hydrodynamic diameter similar to the previous PS formulations (average size: 116 ± 8 nm, PDI ~0.15), demonstrating that the dye presence in the PS’s membrane is not affecting the structure of the nanovesicles.

For assessing tumor internalization MCF10CA1a cancer cells - an aggressive human derived TNBC cell line - were used. These cells are known to overexpress surface p32 and NRP-1 proteins, thus making them a good target for LinTT1 and RPAR CendR peptides. TNBC model was used in vivo because LinTT1 peptide has already been used for early detection and treatment of breast tumors.

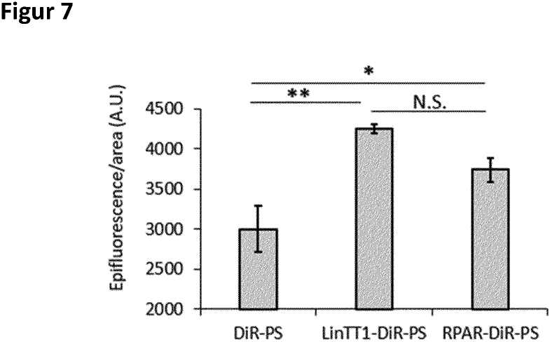

LinTT1-DiR-PS, RPAR-DiR-PS, and DiR-PS were injected i.v. into TNBC mice and live imaging was carried out at 1; 3; 6; 24; and 48 h post-injection (FIG. 6). Targeting with LinTT1 and RPAR peptides increased tumor homing of DiR-PS. LinTT1- and RPAR-DiR-PS were detected in the tumor at 3 h after administration, already, while untargeted DiR-PS were visible starting from 24 h post-injection, only. The highest tumor homing was observed 24 and 48 h after injection for LinTT1-DiR-PS. The integral intensity, assessed by the area under the curve (AUC, FIG. 7), in the tumor at 24 h is approx. 42% higher compared to DiR-PS. The AUC for RPAR-DiR-PS was also significantly higher compared to DiR-PS (approx. 25% higher). After 24 and 48 h LinTT1-, RPAR-, and non-targeted DiR-PS were also observed in the liver as well as in the spleen. This can be explained by the crucial role of these organs in the body clearance of drugs and NPs. 48 h after LinTT1-DiR-PS injection, breast tumors and heart were excised and the microscopic localization of PS was analyzed by fluorescence confocal microscopy. The accumulation of LinTT1-DiR-PS deep inside the tumor parenchyma was visible. Cardiotoxicity is one of the drawbacks of anthracyclines, and, therefore, also the accumulation of LinTT1-DiR-PS in the heart of TNBC-bearing mice was assessed. Significantly lower PS signal levels were observed in the heart tissue in comparison to the tumor cells. The LinTT1 receptor, p32, is also overexpressed in activated macrophages, which play an important role in tumor progression.

In addition, also the LinTT1-DiR-PS co-localization with the CD206 receptor, expressed in pro-tumoral M2 macrophages was measured. It was observed that LinTT1-DiR-PS targeted M2 macrophages in the tumor.

### 3.5. In Vivo Testing - Drug Accumulation in the Tumor

Encouraged by the enhanced LinTT1-targeted PS accumulation effect, the drug accumulation after i.v. administration of LinTT1-UTO-PS, UTO-PS, and free DOX in orthotopic MCF10CA1a tumor bearing mice was studied. The samples were injected, allowed to circulate for 24 h, tumors were collected and analyzed by confocal immunoanalysis.

A significantly higher UTO tumor accumulation in mice injected with LinTT1-UTO-PS in comparison with other samples was found. The UTO fluorescence observed with low co-localization with blood vessels (CD31 staining), suggested that UTO loaded in LinTT1-PS have extravasated and penetrated into the tumor tissue. This result demonstrates that the encapsulation of UTO in LinTT1-PS enhanced the tumor accumulation and penetration of the drug, showing the potential application of our formulation for efficient TNBC treatment.

### 4. Comparison PS-UTO vs. PS-DOX

In order to quantitatively evaluate the differences of DOX and UTO in a PS encapsulation system a direct comparison was performed. UTO and DOX were both encapsulated in PEG-PCL-PS using the film hydration method. The encapsulation and PS formation for this test was performed as described below:

### 4.1 PS Formation

Polymersomes were prepared by dissolving 5 mg of PEG-PCL in 0.3 mL acetone. The acetone was evaporated with nitrogen flow forming a thin polymeric film on the wall of the glass vial. The film was hydrated with 0.5 mL of PBS pH 7.4 previously purged with nitrogen flow, heated for 30 seconds in a 65° C. water bath, and sonicated for 30 seconds. The heating and sonication steps were repeated until the PS were formed and polymer aggregates were not observed in the suspension. The final volume of PS samples was 0.5 mL and the total polymer concentration 10 mg/mL.

### 4.2 Drug Encapsulation

For the PS encapsulation of UTO 50 nM of UTO were dissolved in 100 µL of acetone (0.5 mM concentration) and added to the polymers dissolved in acetone (total amount of polymer - 5 mg, UTO concentration 0.125 mM). The acetone was evaporated to form the polymer/drug film and the PS were formed as described above.

For DOX encapsulation the polymeric film was hydrated with 2 mM DOX solution in PBS pH 7.4 and the PS were formed as described above. The higher DOX concentration is necessary because the DOX encapsulation efficiency is approximately 20 times lower compared to UTO. The used difference leads to a comparable UTO and DOX concentration in the PS.

### 4.3 Purification

UTO-PS and DOX-PS samples were purified using size exclusion chromatography. As a stationary phase agarose beads were used with a diameter of 45-165 µm (Sephadex 4B gel). The height of the Sephadex gel in a column was 8 cm (volume 25.13 mL) giving the dead volume of ~2.5 mL. The PS fraction (0.5 mL) was collected until the turbidity of the solution, indicating the presence of PS, was not observed.

### 4.3 Encapsulation Efficiency (EE)

The UTO and DOX PS-encapsulated amounts were quantified using UV-VIS spectrometry (Thermo Scientific, USA). For UTO quantification, serial dilutions of UTO in MeOH:water 1:1 were prepared and the absorbance at 485 nm was used for calibration. For DOX quantification, serial dilutions of DOX in PBS were prepared and also the absorbance at 485 nm was measured. A linear fit of the data was further used to evaluate the UTO and DOX concentration.

The quantitative results reveal, that after purification the encapsulation efficiency (EE) of UTO-loaded PS was 85% and dramatically higher compared to the 2.3% achievable for doxorubicin. HCl (DOX). This is very surprising based on the similar chemical structures of the different drugs. Consequently, much higher drug loadings can be achieved by using UTO instead of DOX.

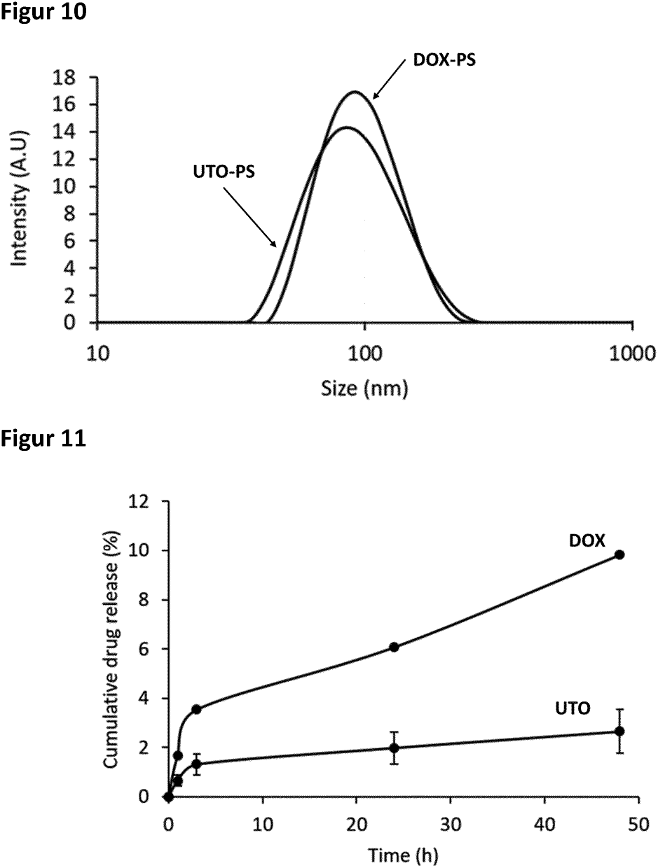

FIG. 10 displays the hydrodynamic diameter of DOX-PS and UTO-PS measured by Dynamic Light Scattering (DLS). Very similar PS sizes are achievable using DOX or UTO. The mean particle size is 92 nm (+- 33) for DOX-PS and 87 nm (+- 37) for UTO-PS.

### 4.3 Drug Release

To further evaluate the UTO and DOX cumulative release behavior from polymersomes, UTO-and DOX-loaded PS were incubated at 37° C. in PBS (0.25 mL) for different time periods (0; 1; 4; 24; and 48 h). The samples were centrifuged using Amicon Ultra centrifugal filters (MWCO 100 kDa) for 20 min at 6,000 g at RT. The fluorescence of the filtrates was measured at 485 nm/535 nm (0.1 s) using a Victor X5 Multilabel Microplate Reader (Perkin Elmer, USA) to quantify the released drug amount.

The results of the drug release are displayed in FIG. 11. It is shown that after 48 h less than 3% of UTO was released from the PS. In comparison to UTO approximately 10% DOX was released from the PS. This finding is a further indicator that UTO is surprisingly the “better” drug in a PS encapsulation system. The drug is encapsulated in higher amounts and the drug is better retained in PS compared to DOX. Such a behavior is very surprising, considering the structural similarities of UTO and DOX. Nevertheless, the better in-vivo efficacy might also be based, at least in part, on the better encapsulation and higher storage stability compared to DOX.

What is claimed:

1) Drug delivery system at least comprising a drug encapsulated in a polymeric nanovesicle, characterized in that the drug component is an anthracycline derivative according to the formula I 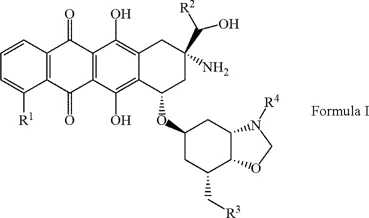
wherein R¹ is selected from the group consisting of H, F, —OMe and —OEt;
R² is selected from the group consisting of H, —OMe, methyl and ethyl;
R³ is selected from the group consisting of H, methyl and ethyl, and
R⁴ is H or a protecting group;
wherein the polymersome is formed by polymers comprising PEG, PLA, PCL, PTMC or PTMB building blocks or combinations thereof, wherein the polymersome polymers are, at least in part, functionalized by chemically attaching via a linker group L a targeting moiety, and wherein the targeting moiety is selected from the group consisting of antibodies, peptides, aptamers or mixtures thereof.

2) The drug delivery system according to claim 1, wherein the targeting moiety is a peptide and the peptide is a tumor penetrating peptide selected from the group of consisting of CendR peptides, iRGD (CRGDKGPDC), LyP-1 (CGNKRTRGC), RPAR (RPARPAR), TT1 (CKRGARSTC), LinTT1 (AKRGARSTA), iNGR (CRNGRGPDC), tLyp-1 (CGNKRTR) or precursors thereof.

3) The drug delivery system according to claim 1, wherein the polymersome comprises di-block PEG-copolymers, wherein the second block is selected from the group consisting of PLA, PCL, PTMC and PTMBP.

4) The drug delivery system according to claim 1, wherein the polymersome consists of PEG-PCL diblock-copolymers, and wherein the different polymer blocks have a weight ratio of calculated as PEG-segment weight divided by PCL-segment weight, is larger or equal 0.1 and smaller or equal 5.

5) The drug delivery system according to claim 1, wherein in formula I R¹ = H, R² is CH₃, R³ = H, and R⁴ is a protection group.

6) The drug delivery system according to claim 5, wherein R⁴ is acetyloxymethyl carbamate.

7) The drug delivery system according to claim 1, wherein the linker group L is maleimide.

8) The drug delivery system according to claim 1, wherein the polymersome has a molar ratio of peptide modified polymer chains to the total number of polymers chains calculated as number of peptide-modified polymer chains divided by total number of polymer chains, is larger or equal to 0.01 and smaller or equal to 0.4.

9) The drug delivery system according to claim 1, wherein the polymersome has a molar ratio of peptide modified polymer chains to the total number of polymers chains calculated as number of peptide-modified polymer chains divided by total number of polymer chains, is larger or equal to 0.05 and smaller or equal to 0.1.

10) The drug delivery system according to claim 1, wherein the polymeric nanovesicle has a concentration of the drug which is larger or equal to 20 µM and smaller or equal to 500 µM.

11) The drug delivery system according to claim 1, wherein the drug loaded polymeric nanovesicle has a polydispersity index which is larger or equal to 0.01 and smaller or equal to 0.25.

12) A process for the production of an anthracycline derivative loaded, targeted polymersome drug delivery system according to claim 1, characterized in that the drug according to formula I is encapsulated in the polymeric nanovesicle by a thin-film hydration step.

13) The process according to claim 12, wherein the drug loaded polymeric nanovesicle is subjected in a further step to a size-exclusion chromatographic step.

14) A pharmaceutical composition comprising the drug delivery system according to claim 1, in a pharmaceutically acceptable solvent.

15) A method of treating cancer comprising the pharmaceutical composition according to claim 14.
